(19) United States
(12) Patent Application Publication
(10) Pub. No.: US 2006/0228719 A1
(43) Pub. Date: Oct. 12, 2006

(54) Method for imaging an array of microspheres using specular illumination

(75) Inventors: Martin C. Kaplan, Rochester, NY (US); Krishnan Chari, Fairport, NY (US); Samuel Chen, Penfield, NY (US); Douglas L. Vizard, Durham, CT (US)

Correspondence Address: Pamela R. Crocker;Patent Legal Staff, Eastman Kodak Company, 343 State Street, Rochester, NY, 14650-2201, US

(73) Assignee: Eastman Kodak Company

(21) Appl. No.: 11/103,763

(22) Filed: Apr. 12, 2005

(51) Int. Cl. C12Q 1/68; G06K 9/00; G01N 33/551

(52) U.S. Cl. 435/6; 436/524; 382/128

(57) ABSTRACT

A specular light source and camera for detecting and quantifying the presence of biological probes, which contain a colorant with spectral properties that depend upon the angle of illumination and angle of imaging, that indicate the presence of specific chemical moieties within a biological system.

### FIELD OF THE INVENTION

The present invention relates in general to molecular biological systems and more particularly to a means to simplify the detection process for colored bead random microarrays.

### BACKGROUND OF THE INVENTION

Ever since their invention in the early 1990s (Science, 251, 767-773, 1991), high-density arrays formed by the spatially addressable synthesis of bioactive probes on a 2-dimensional solid support have enhanced and simplified the process of biological research and development. The key to current microarray technology is deposition of a bioactive agent at a single spot on a microchip in a “spatially addressable” manner.

Current technologies have used various approaches to fabricate microarrays. For example, U.S. Pat. No. 5,412,087, inv. McGall et al., issued on May 2, 1995 and U.S. Pat. No. 5,489,678, inv. Fodor et al., issued Feb. 6, 1996, demonstrate the use of a photolithographic process for making peptide and DNA microarrays. The patent is directed to the use of photolabile protecting groups to prepare peptide and DNA microarrays through successive cycles of deprotecting a defined spot on a 1 cm.-×1 cm chip by photolithography, then flooding the entire surface with an activated amino acid or DNA base. Repetition of this process allows construction of a peptide or DNA microarray with thousands of arbitrarily different peptides or oligonucleotide sequences at different spots on the array. This method is expensive.

An ink jet approach has been used by others (e.g., Papen et al., U.S. Pat. No. 6,079,283, issued Jun. 27, 2000, U.S. Pat. No. 6,083,762; issued, Jul. 4, 2000 and U.S. Pat. No. 6,094,966, issued Aug. 1, 2002) to fabricate spatially addressable arrays, but this technique also suffers from high manufacturing cost in addition to the relatively large spot size of 40 to 100 μm. Because the number of bioactive probes to be placed on a single chip usually runs anywhere from 1000 to 100000 probes, the spatial addressing method is intrinsically expensive regardless how the chip is manufactured.

An alternative approach to the spatially addressable method is the concept of using fluorescent dye-incorporated polymeric beads to produce biological multiplexed arrays. U.S. Pat. No. 5,981,180, inv. Chandler et al., issued Nov. 9, 1999 discloses a method of using color coded beads in conjunction with flow cytometry to perform multiplexed biological assay. Microspheres conjugated with DNA or monoclonal antibody probes on their surfaces were dyed internally with various ratios of two distinct fluorescence dyes. Hundreds of “spectrally addressed” microspheres were allowed to react with a biological sample and the “liquid array” was analyzed by passing a single microsphere through a flow cytometry cell to decode sample information.

U.S. Pat. No. 6,023,540, inv. Walt et al., issued Feb. 8, 2000 discloses the use of fiber-optic bundles with pre-etched microwells at distal ends to assemble dye loaded microspheres. The surface of each spectrally addressed microsphere was attached with a unique bioactive agent and thousands of micro spheres carrying different bioactive probes combined to form “beads array” on pre-etched microwells of fiber optical bundles.

More recently, an optically encoded microsphere approach was accomplished by using different sized zinc sulfide-capped cadmium selenide nanocrystals incorporated into microspheres (Nature Biotech. 19, 631-635, (2001)). Given the narrow band width demonstrated by these nanocrystals, this approach expands the spectral barcoding capacity in microspheres.

Even though the “spectrally addressed microsphere” approach does provide an advantage in terms of its simplicity over the old fashioned “spatially addressable” approach in microarray making, recent improvements in the art make the manufacture and use of random microarrays less difficult and less expensive.

A coating technology is described in US Patent Application No. 2003/0170392 A1 to prepare a microarray on a substrate that need not be pre-etched with microwells or premarked in any way with sites to attract the microspheres. Using unmarked substrates, or substrates that need no pre-coating preparation, provides a huge manufacturing advantage compared to the existing technologies. Color addressable mixed beads in a dispersion can be randomly distributed on a receiving layer that has no wells or sites to attract the microspheres. This method provides a microarray that is less costly and easier to prepare than those previously disclosed because the substrate does not have to be modified even though the microspheres remain immobilized on the substrate, where the bead surfaces are exposed to facilitate easier access of the analyte to probes attached to the surfaces of the beads.

US Patent Application No. 2003/0068609 A1 discloses a coating composition and technology for making a microarray on a substrate that does not have specific sites capable of interacting physically or chemically with the microspheres. The substrate need not be pre-etched with microwells or premarked in any way with sites to attract the microspheres. Upon coating the composition on a substrate, the microspheres become immobilized in the plane of coating and form a random pattern on the substrate. Using unmarked substrates or substrates that need no pre-coating preparation provides a manufacturing means that is less costly and easier to prepare than those previously disclosed because the substrate does not have to be modified compared to the existing technologies. A composition allows color addressable mixed beads to be randomly distributed on a substrate that has no wells or sites to attract the microspheres. A method of making a random array of microspheres using enzyme digestion to expose surfaces of the microspheres is described in US Patent Application No. 2003/0224361 A1. Enzyme digestion can be easily controlled to expose the desired amount of microsphere and the enzyme, a protease, is readily available and economical to obtain.

A method of manufacturing and detecting colored microarrays is described in US Patent Application No. 2004/0106114 A1. During the manufacture of the microspheres, an optical bar code is generated of the colorants associated with the microspheres and stored in a digital file. The biologically/chemically active region of a support treated with the microspheres is scanned with a high-resolution color scanner to produce a color map of the locations of the randomly dispersed set of one color of microspheres. A digital file of the color map produced is linked the digital file of the color map with the support. After the microarray is exposed to an analyte, the microarray is scanned by a monochrome scanner and a bead map of the microbeads is produced. The map is linked through the digital file to the location of the colored beads when the support was manufactured.

There exists a need in the art for improved methods of detection which will make the manufacture and use of micro arrays less difficult and less expensive.

### SUMMARY OF THE INVENTION

A random or ordered array of colored beads, preferably arrayed on a substrate, is imaged using a specular light source and an imaging device, such as a digital camera. The beads are treated to act as probes, which can attach to various materials, such as proteins or genetic material, in a biological sample. More than one color of bead is present, with beads of different colors treated to probe for different materials, such as proteins or genetic material. The bead colorant has different spectral reflectance properties depending on the angle of illumination and angle of imaging. Colorants can include multilayer dichroic filters and cholesteric liquid crystals. The spectral reflectance of each bead, which is termed the “color” of the bead, is determined by imaging the beads at several wavelengths. The specularity of the illumination and imaging systems limits the range of angles that may be used to illuminate and image the micro arrays, and thereby limits the spectral variability of the colorant. This improves the ability to distinguish among different bead colorants (and thus differently colored beads). This allows the use of a large number of distinguishable colorants, permitting the use of a large number of distinct bead colors, which results in a process that allows simultaneously probing for a large number of different proteins or genetic materials.

Beads are also treated with fluorescent and/or chemiluminescent markers to indicate the presence and/or quantity of the protein or genetic material. For chemiluminescent markers, the beads are imaged during the interaction of the bead with the sample material, detecting the spatial position of the chemiluminescing beads. For fluorescent markers, the tunable light source is tuned to wavelengths that stimulate fluorescence, and an image of the beads is taken through a filter that blocks the stimulating wavelength but transmits the fluorescent emitted wavelengths. Either before or after measuring the chemiluminescence or fluorescence, the tunable light source is tuned to several wavelengths, or wavelength ranges, and the digital camera captures an image of the beads, usually with the fluorescent filter removed, at each wavelength. Images are captured with different spectral responses of the illumination/camera system, such as by a wavelength tunable illuminator, or filters in the camera system, or between the beads and the camera. Angle dependent spectral reflectance is typical of many materials that involve optical interference effects.

The presence of protein/genetic material at probes containing fluorescent/chemiluminescent signal is indicated by the spatial position of the chemiluminescent/fluorescent signal. The spectrally determined “color” of the bead identifies the type of protein/genetic material for which the bead was prepared to probe, and thus the type of protein/genetic material that has been detected. There are several advantages to use of this invention. The spectral variation of the colorant is limited by the specularity of the illumination and the limited range of angles used in imaging the beads, allowing for an improved ability to distinguish among the bead colorants, improving the ability to distinguish one color of bead from another. This improves the use of random arrays of beads, which are less expensive to manufacture than carefully ordered arrays. Use of dichroic filters allows use of a large number of distinguishable colorants, whereby a large number of probes can be simultaneously used to analyze biological material.

### BRIEF DESCRIPTION OF THE DRAWINGS

The foregoing and other objects, features, and advantages of the invention will be apparent from the following more particular description of the embodiments of the invention, as illustrated in the accompanying drawings. The elements of the drawings are not necessarily to scale relative to each other.

FIG. 1 is a diagram of the composition of a microarray.

FIG. 2 is a diagram of a method of imaging the microarray.

### DETAILED DESCRIPTION OF THE INVENTION

The foregoing and other objects, features, and advantages of the invention will be apparent from the following more particular description of the embodiments of the invention, as illustrated in the accompanying drawings. The elements of the drawings are not necessarily to scale relative to each other.

The present invention teaches a method for imaging a random or ordered array of microspheres, also referred to as “beads”, immobilized in a coating on a substrate. The microspheres are desirably formed to have a mean diameter in the range of 1 to 50 microns; more preferably in the range of 3 to 30 microns and most preferably in the range of 5 to 20 microns. It is preferred that the concentration of micro spheres in the coating is in the range of 100 to a million per cm², more preferably 1000 to 200,000 per cm² and most preferably 10,000 to 100,000 per cm².

Although microspheres or particles having a substantially curvilinear shape are preferred because of ease of preparation, particles of other shape such as ellipsoidal or cubic particles may also be employed. Suitable methods for preparing the particles are emulsion polymerization as described in “Emulsion Polymerization” by I. Piirma, Academic Press, New York (1982) or by limited coalescence as described by T. H. Whitesides and D. S. Ross in J. Colloid Interface Science, vol. 169, pages 48-59, (1985). The particular polymer employed to make the particles or microspheres is a water immiscible synthetic polymer that may be colored. The preferred polymer is any amorphous water immiscible polymer. Examples of polymer types that are useful are polystyrene, poly(methyl methacrylate) or poly(butyl acrylate). Copolymers such as a copolymer of styrene and butyl acrylate may also be used. Polystyrene polymers are conveniently used.

The beads are treated to act as “probes”, by the attachment of bioactive agents to the surface of chemically functionalized microspheres. This can be performed according to the published procedures in the art (Bangs Laboratories, Inc, Technote #205). Some commonly used chemical functional groups include, but are not limited to, carboxyl, amino, hydroxyl, hydrazide, amide, chloromethyl, epoxy, aldehyde, etc. Examples of bioactive agents or probes include, but are not limited to, oligonucleotides, DNA and DNA fragments, PNAs, peptides, antibodies, enzymes, proteins, and synthetic molecules having biological activities.

The beads are also treated with a insoluble colorant, or combination of colorants, whose spectral reflectance properties depend upon the angle of illumination and the angle of imaging. The colorant, or dye, is not dissolved during array coating or subsequent treatment or the beads. Suitable colorants may be oil-soluble in nature. It is preferred that the colorants are non-fluorescent when incorporated in the microspheres. Methods for coating beads are broadly described by Edward Cohen and Edgar B. Gutoff in Chapter 1 of “Modern Coating And Drying Technology”, (Interfacial Engineering Series; v.1), (1992), VCH Publishers Inc., New York, N.Y. For a single layer format, suitable coating methods may include dip coating, rod coating, knife coating, blade coating, air knife coating, gravure coating, forward and reverse roll coating, and slot and extrusion coating. Beads are also treated with fluorescent and/or chemiluminescent markers to indicate the presence and/or quantity of the protein or genetic material. The location of the fluorescent and/or chemiluminescent markers are matched with the location of the colored beads to identify the probes that interacted with the biological material.

The microarray consists of two or more types of beads, each of which is treated to react with a specific moiety and has a unique color. The distribution or pattern of the microspheres on the substrate is either arrayed or entirely random. The microspheres are not attracted or held to sites that are pre-marked or predetermined on the substrate. The term “random distribution”, as used herein, means a spatial distribution of elements showing no preference or bias. Randomness can be measured in terms of compliance with that which is expected by a Poisson distribution. The surface of the microspheres bear capture agents, or probes, which are readily accessible to analytes with which they come in contact.

During, or after, exposure to a biological sample, a random or ordered array of colored beads, preferably arrayed on a substrate, is imaged by illuminating the microarray using a specular light source 10 and an imaging device 15, such as a color camera as illustrated in FIG. 2. The specularity of the illumination and imaging systems limits the range of angles that may be used to illuminate and image the microarrays. This limits the spectral variability of the colorant and improves the ability to distinguish among different bead colorants (and thus differently colored beads). A large number of distinguishable colorants can be used, allowing the use of a large number of distinct bead colors. This results in a process that allows simultaneously probing for a large number of different proteins or genetic materials. The specular light source 10 may include a wavelength tunable illuminator, or a broad-spectrum illuminator used in combination with filters to control the wavelength, or range of wavelengths, within the system. When chemiluminescent markers are used, the beads are imaged during the interaction of the bead with the sample material, allowing the spatial position of the chemiluminescing beads to be determined. When fluorescent markers are used, the tunable light source is tuned to wavelengths that stimulate fluorescence, and an image of the beads is taken through a filter that blocks the stimulating wavelength but transmits the fluorescent emitted wavelengths. Either before or after measuring the chemiluminescence or fluorescence, the tunable light source is tuned to several wavelengths, or wavelength ranges, and an image of the beads is collected, usually with the fluorescent filter removed, at each wavelength. Alternatively, a filter, or combination of filters, can be used to select for the desired wavelengths of light. The spectral reflectance of each bead, which is termed the “color” of the bead, is determined by imaging the beads at several wavelengths.

The presence of biological material at probes containing a fluorescent/chemiluminescent signal is indicated by the spatial position of the chemiluminescent/fluorescent signal. The spectrally determined “color” of the bead at the location of the chemiluminescent/fluorescent signal identifies the bead and the corresponding moiety for which the bead was prepared to probe.

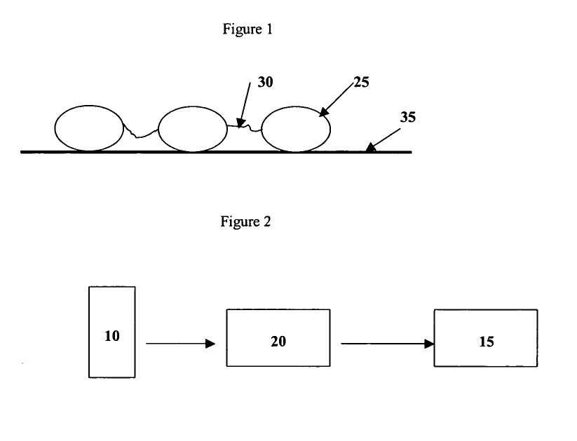

FIG. 1 shows a diagram of a microarray described in this invention. The microarray 20 is composed of colored beads 25, or microspheres, dispersed preferably in a coating 30 on a substrate 35. The beads 25 contain a biological/chemical probe 40 and at least one colorant 45 that has different spectral reflectance properties depending on the angle of illumination and angle of imaging.

FIG. 2 shows a diagram of a method of imaging the microarray 20 by illuminating the microarray 20 using a specular light source 10 and an imaging device 15, such as a color camera. Depending upon the nature of the beads used, imaging may occur during, or after, exposure to a biological sample.

All documents, patents, journal articles and other materials cited in the present application are hereby incorporated by reference.

The invention has been described in detail with particular reference to a presently preferred embodiment, but it will be understood that variations and modifications can be effected within the spirit and scope of the invention. The presently disclosed embodiments are therefore considered in all respects to be illustrative and not restrictive. The scope of the invention is indicated by the appended claims, and all changes that come within the meaning and range of equivalents thereof are intended to be embraced therein.

### PARTS LIST

10 specular light source
15 imaging device
20 microarray
25 colored beads, or microspheres
30 coating
35 substrate
40 biological/chemical probe
45 colorant

1. A method of imaging an array of colored beads, the method comprising the steps of:
exposing an array of color coded beads containing a multiple of colors to a spectrum of specular illumination from a light source, wherein each bead contains a colorant whose spectral reflectance properties depend upon the angle of illumination and the angle of imaging, and each colorant is coded to identify a probe for detecting a specific chemical moiety, a specific biochemical moiety, or a combination thereof, and at least one of the colored beads contains a marker indicating the presence of biological material;
changing the spectrum of said specular illumination in the system; and
capturing an image of the beads at each spectrum using an imaging device.

2. The method according to claim 1 wherein the color of a bead is coded to identify a probe for detecting a specific chemical moiety, a specific biochemical moiety, or a combination thereof.

3. The method according to claim 2 wherein the biochemical moiety is a protein.

4. The method according to claim 2 wherein the biochemical moiety is genetic material.

5. The method according to claim 1 wherein the array is spatially random.

6. The method according to claim 1 wherein the array is spatially ordered.

7. The method according to claim 1 wherein the array is supported on a substrate.

8. The method according to claim 1 wherein the image is captured using a camera or positional scanning.

9. The method according to claim 1 wherein the wavelength of light in the system is changed using a wavelength tunable illuminator.

10. The method according to claim 1 wherein the wavelength of light in the system is changed by passing the light through a filter, or combination 10 of filters between the beads and the imaging system.

11. The method according to claim 10 wherein the filter, or combination of filters, are part of the imaging device.

12. The method according to claim 1 wherein at least one color of bead is treated to act as a probe for identifying the moiety recognized by said probe, and said at least one color of bead contains a fluorescent or chemiluminescent marker to indicate the presence, quantity, or combination thereof for a chemical moiety in a biological sample.

13. The method according to claim 12 wherein at least one color of bead contains at least one fluorescent marker and at least one color of bead contain at least one chemiluminescent marker.

14. The method according to claim 1 wherein at least one color of bead is treated to act as a probe for identifying the moiety recognized by said probe, and said at least one color of bead contains a fluorescent or chemiluminescent marker to indicate the presence, quantity, or combination thereof for a protein, genetic material or other material of biological origin.

15. The method according to claim 14 wherein at least one color of bead contains at least one fluorescent marker and at least one color of bead contain at least one chemiluminescent marker.

16. The method according to claim 1 wherein at least one color of bead contains a chemiluminescent marker, and the bead is imaged during the interaction of the bead with the biological material, thereby detecting the spatial position of the chemiluminescing bead.

17. The method according to claim 1 wherein at least one color of bead contains a fluorescent marker, the tunable light source is tuned to a stimulating wavelength or wavelength range that stimulates the fluorescence, and the bead is imaged through a filter that blocks the stimulating wavelength or wavelength range but transmits the fluorescent emitted wavelengths.

18. The method according to claim 1 wherein beads of at least two colors are present and at least two fluorescent markers are present that fluoresce at different wavelength ranges, the light spectrum is tuned to a first optimum wavelength, or wavelength range, that stimulates fluorescence of a first fluorescent molecule and an image is collected, and the light spectrum is tuned to an optimum wavelength, or wavelength range, that stimulates fluorescence for each additional fluorescent molecule present and an image is collected.

19. The method according to claim 1 wherein the image of the array is captured at each wavelength or wavelength range with and without a fluorescence blocking filter.

20. The method according to claim 1 wherein the light source allows imaging at non-visible wavelengths.
